(19) United States
(12) Patent Application Publication
(10) Pub. No.: US 2011/0274727 A1
(43) Pub. Date: Nov. 10, 2011

(54) DEPIGMENTING TOPICAL COMPOSITIONS AND THEIR USES

(75) Inventors: Philippe Andres, Peymeinade (FR); Isabelle Pelisson, Vallauris (FR)

(73) Assignee: GALDERMA RESEARCH & DEVELOPMENT, Biot (FR)

(21) Appl. No.: 13/126,215

(22) PCT Filed: Oct. 28, 2009
(86) PCT No.: PCT/EP2009/064238

§ 371 Date: Jul. 28, 2011

Related U.S. Application Data
(60) Provisional application No. 61/109,061, filed on Oct. 28, 2008.

(30) Foreign Application Priority Data
Jun. 5, 2009 (FR) 0953745

(51) Int. Cl. A61K 9/00; A61P 17/12; A61Q 19/02; A61K 8/34; A61P 17/00

(52) U.S. Cl. 424/400; 424/62

(57) ABSTRACT

Depigmenting topical compositions and their uses are described. In particular, the described compositions can provide improved effectiveness in the dermatological treatment of pigmentation, without disadvantages experienced with other known compositions. The described topical compositions can comprise, in a physiologically acceptable medium, from 3% to 10% of rucinol or one of its salts. Also described, are methods of use treating pigmentation disorders of the skin with the described compositions.

The present invention relates to the use of rucinol or one of its salts, as sole pharmaceutical active principle, at a concentration of 3 to 10% by weight, preferably of 3 to 5% by weight and more preferably of 5% by weight, with respect to the total weight of the composition, in a topical composition, the said composition being intended for the treatment of hyperpigmentary disorders of the human skin.

Hyperpigmentation of the skin is a common disorder expressed by the appearance of liver spots or coloured blemishes on skin fragments. Hyperpigmentation occurs during accumulations of melanin in the skin, conferring nonuniformity on the skin. The pigmented patches can appear on any part of the body, in particular on the backs of the hands, the face, the neckline and bald heads.

Several factors can contribute to the development of hyperpigmented lesions, the most frequently mentioned being genetic predisposition, hormones, exposure to the sun and skin aging. In addition, pigmented patches can appear following attacks on or inflammation of the skin. An increase in the production of melanin can thus be brought about by a cutaneous inflammatory process, for example after trauma, inflammatory eruptions, or other phenomena, such as skin irritation.

A common form of hyperpigmentation among disorders of pigmentation consists of age spots or sun spots. They are due to damage from the sun and usually appear on the backs of the hands and arms, on the neckline or on the face. These spots are darker than freckles or ephelides, and persist into the winter.

Consequently, there thus exists a real need for an effective and risk-free treatment of the symptoms of photoaging, in particular the hyperpigmentary blemishes brought about by exposure to ultraviolet radiation.

Chloasma or melasma patches are more extensive than age spots and are localized on the face. They are often but not solely related to hormonal changes. Pregnancy, for example, can trigger the excess production of melanin, which causes the “mask of pregnancy”.

The changes in colour of the skin can result from external causes, such as, for example, skin diseases, such as acne, or skin lesions. Freckles are also small brown spots which can appear anywhere on the body but are commonest on the face and arms. Freckles are often an inherited characteristic.

Postinflammatory hyperpigmentation (PIHP) is also a frequent disorder of pigmentation which results from various cutaneous disorders and also from therapeutic interventions. This excess in colouring of the skin can be attributed to infections, allergic reactions, mechanical injuries, such as an abrasion or a scar, reactions to medicaments, such as tetracycline, bleomycin, doxorubicin, 5-fluorouracil, busulfan, arsenic compounds, silver, gold, antimalarial medicaments, hormones and clofazimine, phototoxic eruptions, a scar, a trauma, such as a burn, and also the consequence of inflammatory diseases, such as acne, lichen planus, lupus erythematosus, atopic dermatitis and cutaneous T cell lymphoma.

PHIP is commoner in dark phototypes, such as noncaucasian skin, in particular Asian, black or mixed race skin.

The depigmenting agents or lightening agents currently used in the form of topical compositions make it possible to reduce the density of melanin in the epidermis and possibly in the dermis. These agents are generally absorbed through the lower layers of the epidermis and slow down the formation of melanin.

Hydroquinone is a depigmenting agent, as are its derivatives, such as benzyloxyphenol and hydroquinone monobenzyl ether. Hydroquinone is the reference depigmenting product conventionally used for more than 30 years worldwide for the treatment of hyperpigmentary disorders, such as melasma or lentigines. This depigmenting agent is used mainly at 2 or 4%, depending on the country, in creams, gels or lotions and is applied twice daily, in the morning and in the evening.

However, these agents exhibit several disadvantages: hydroquinone is unstable in an alkaline medium and is oxidized in the form of quinone, which gives a brownish colour to the compositions comprising it; moreover, hydroquinone is irritating; it can also induce hypersensitivity reactions and, in some rare cases, ochronosis (disorder of the colouring of the skin, characterized by the appearance of blue-black blemishes); hydroquinone is also suspected of being carcinogenic; hydroquinone monobenzyl ether is not correctly metabolized when it is absorbed through the skin and brings about irreversible depigmentations. Methoxyphenol, a hydroquinone ether, which exhibits the disadvantage of being not very soluble in water and difficult to incorporate in cosmetic or dermatological formulations, is also known.

A depigmenting composition comprising hydroquinone, retinoic acid and dexamethasone has been described (U.S. Pat. No. 3,856,934) but this composition is also irritating and can, in the most extreme cases, bring about itching sensations.

Various products of the vitamin C, fruit acid and sunscreen type have been developed for treating these pigmentation problems but the majority of them include unstable mixtures which risk replacing the dark patches with light patches, which are more visible, and the majority have a fairly slow action.

There thus exists a need to treat hyperpigmentation patches and to remove the skin defects general due to the deposition of excessive amounts of melanin.

The products Iklen® Cream and Serum, sold by Merck Medication Familiale, comprising 0.3% of rucinol, are used as depigmenting products in some hyperpigmentary disorders, such as lentigines and melasma. These cosmetic products do not make it possible to obtain a lasting and satisfactory depigmentation of the pigmentary blemishes of the skin. To date, no depigmenting product on the market comprises more than 0.3% of rucinol.

On consideration of the above, a problem which the invention intends to solve is that of producing a composition which shows an improved effectiveness in the dermatological treatment of pigmentation, without the disadvantages of the compositions of the prior art.

A first subject-matter of the invention is a topical composition intended for the treatment of disorders of pigmentation of the skin, characterized in that it comprises, in a physiologically acceptable medium, rucinol or one of its salts, as sole pharmaceutical active principle, present at a concentration of at least 3% by weight, preferably ranging from 3 to 10% by weight, more preferably from 3 to 5% by weight and more preferably still of 5% by weight, with respect to the total weight of the composition.

Topical composition is understood to mean a composition intended to be applied to the skin and/or mucous membranes.

Physiologically acceptable medium is understood to mean a medium compatible with the skin, mucous membranes and/or superficial body growths.

A second subject-matter of the invention is the use of a topical composition comprising rucinol or one of its salts, as sole pharmaceutical active principle, at a concentration of at least 3% by weight, preferably at a concentration of 3 to 10% by weight, more preferably of 3 to 5% by weight and more preferably still of 5% by weight, with respect to the total weight of the composition, in the preparation of a medicament intended for the treatment of disorders of pigmentation of human skin.

A further subject-matter of the invention is a method for the dermatological treatment of skin pigmentation, comprising the administration of a composition according to the invention to an individual to be treated.

Rucinol is also known as lucinol or 4-(n-butyl)resorcinol.

Rucinol salts is understood to mean in particular salts formed with a pharmaceutically acceptable base, in particular an inorganic base, such as sodium hydroxide, potassium hydroxide and ammonia, or an organic base, such as lysine, arginine or N-methylglucamine, but also the salts formed with fatty amines, such as dioctylamine, aminomethyl propanol and stearylamine.

Preferably, rucinol will be used.

Surprisingly, the Applicant Company has discovered that a precise amount of rucinol, used as sole active agent, in a topical composition makes it possible to obtain an optimum depigmenting activity while retaining good tolerance of the composition by the skin and thus to limit side effects, such as irritation. This activity is obtained when the rucinol is present on its own in the topical composition at between 3 and 10% by weight, with respect to the total weight of the composition, and preferably between 3 and 5% by weight and more preferably at 5% by weight, with respect to the total weight of the composition.

This is because the Applicant Company has discovered, surprisingly, that the compositions comprising from 3 to 10% of rucinol, used as sole active agent, and preferably from 3 to 5% of rucinol make it possible to obtain much greater activities than the product comprising 4% of hydroquinone, the product conventionally used to treat hyperpigmentary disorders. Given that the product comprising 0.3% of rucinol already existing on the market shows an activity identical to that obtained with the reference product comprising hydroquinone, nothing presaged that the significant increase (at least 10 fold) in the concentration of rucinol in the composition would make it possible to obtain greater effectiveness with regard to hyperpigmentry disorders, such as melasma, while maintaining as good tolerance as the product sold at a lower concentration (0.3%). Thus, the compositions according to the invention make it possible not only to be alternatives to the use of hydroquinone but, in addition, to obtain better results in terms of effectiveness.

The topical compositions according to the invention thus make it possible to reduce local hyperpigmentation of the skin. Specifically, they produce a depigmentation of the skin area on which they are applied.

“Depigmentation” is understood to mean to obtain a decolouration of a hyperpigmented skin area until a colour is obtained similar or close to that of the neighbouring skin.

The compositions of the invention are particularly well suited to the treatment of disorders of pigmentation, such as:

melasma or chloasma,

lentigines or senile lentigo,

vitiligo,

freckles or ephelides,

actinic keratosis,

flat pigmented seborrhoeic warts,

postinflammatory hyperpigmentation, in particular due to infections, allergic reactions, mechanical injuries (such as an abrasion), reactions to medicaments (such as tetracycline, bleomycin, doxorubicin, 5-fluorouracil, busulfan, arsenic compounds, silver, gold, antimalarial medicaments, hormones and clofazimine), phototoxic eruptions, a scar, a trauma (such as a burn) and also the consequence of inflammatory skin diseases (such as acne, psoriasis, rosacea, lichen planus, lupus erythematosus, atopic dermatitis and cutaneous T cell lymphoma);

naevi,

genetically determined hyperpigmentation,

hyperpigmentation of metabolic origin.

The Applicant Company has carried out a comparative study of the effectiveness and tolerance of topical compositions comprising various concentrations of rucinol. The compositions according to the invention respectively comprise 3 and 5% of rucinol; they are compared with the composition comprising 0.3% of rucinol, a concentration conventionally used in cosmetic products, and also with the composition comprising 4% of hydroquinone (product Eldoquin Forte®).

This study involved 48 subjects affected by melasma, divided into groups of 16 patients per product tested. 2 mg/cm² of each product comprising rucinol at 3 or 5% or else the reference product comprising 4% of hydroquinone are applied to an area of 25 cm² of injured skin on the cheek of a patient. The product comprising 0.3% of rucinol is applied to a symmetrical lesion on the other half of the face.

The products were applied once daily, 5 days per week, for 12 weeks.

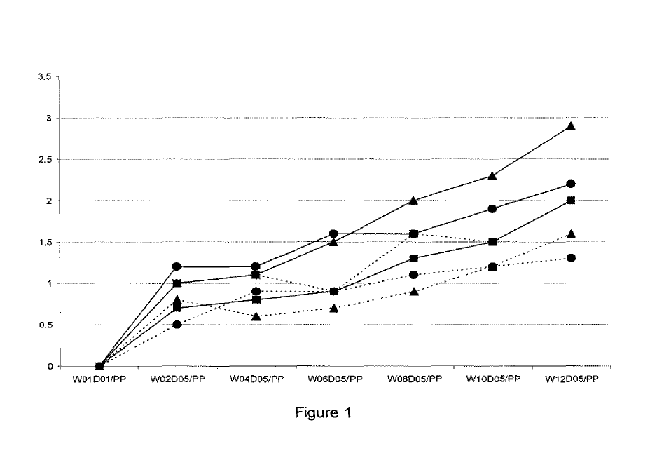

The clinical evaluation of the effectiveness of the product tested was carried out by the measurement of the brightness (L*) (FIG. 1). The colorimetric parameter L*, representing the brightness (0=black, 100=white), is measured at the end of each week using the Konica Minolta CM2600d spectrocolorimeter.

The results obtained during the clinical study are given in FIG. 1, which represents the variations in L* measured each week, with respect to the measurement of L* obtained originally before the 1st application, for each product on the face of each patient.

The curve (▴ as continuous line) represents the mean values of the variation in brightness obtained for the 16 patients with the composition comprising 5% of rucinol, compared with the composition comprising 0.3% of rucinol on the other cheek (▴ as dots).

The curve ( as continuous line) represents the mean values of the variation in brightness obtained for the 16 patients with the composition comprising 3% of rucinol, compared with the composition comprising 0.3% of rucinol on the other cheek ( as dots).

The curve (▪ as continuous line) represents the mean values of the variation in brightness obtained for the 16 patients with the composition comprising 4% of hydroquinone (product Eldoquin Forte), compared with the composition comprising 0.3% of rucinol on the other cheek (▪ as dots).

This study shows a marked increase in the brightness with the compositions comprising 3 and 5% of rucinol in comparison with the compositions comprising 0.3% of rucinol or 4% of hydroquinone. The three curves obtained with the three compositions comprising 0.3% of rucinol are virtually identical and are not significantly different from that obtained with the composition comprising 4% of hydroquinone. This study demonstrates a better depigmenting activity on the part of the compositions according to the invention, compared with the composition comprising 0.3% of rucinol and also with the composition comprising 4% of hydroquinone. And, contrary to all expectations, this study also demonstrated that the compositions according to the invention tested are very well tolerated; specifically, the side effects are not significantly enhanced by the increase in the concentration of rucinol.

The compositions of the invention can additionally comprise any additive conventionally used in the pharmaceutical and dermatological fields which is compatible with rucinol or its salts.

Of course, a person skilled in the art will take care to choose this or these optional additives and/or their amounts so that the advantageous properties of the composition according to the invention are not, or not substantially, detrimentally affected.

Topical route is understood to mean an application on the skin and/or mucous membranes.

The compositions of the present invention can be provided in any formulation form normally used for a topical application, in particular in the liquid, pasty or solid form and more particularly in the form of ointments, of aqueous, aqueous/alcoholic or oily solutions, of dispersions of the lotion type, of aqueous, anhydrous or lipophilic gels, of powders, of impregnated pads, of syndets, of wipes, of sprays, of patches, of foams, of sticks, of shampoos, of compresses, of washing bases, of emulsions with a liquid or semiliquid consistency of the milk type, obtained by dispersion of a fatty phase in an aqueous phase (Oil in Water) or vice-versa (Water in Oil), or of suspensions or emulsions with a soft, semi-liquid or solid consistency of the cream, gel or salve type. It can also be provided in the form of suspensions of microspheres or nanospheres or of lipid or polymeric vesicles or of polymeric or gelled patches which make possible controlled release. This composition by the topical route can be provided in the anhydrous form, in the aqueous form or in the form of an emulsion or also of microemulsions, of microcapsules, of microparticles or of vesicular dispersions of ionic and/or nonionic type.

Advantageously, the composition is provided in the form of a salve, of a cream, of a lotion or of a gel.

### EXAMPLES OF FORMULATIONS

Various real topical formulations for a composition according to the invention have been illustrated in these examples.

### Example 1

A depigmenting cream for the skin of oil-in-water emulsion type is prepared, which cream comprises (% by weight):

| | |
|---|---|
| Rucinol | 3% |
| Glycerol stearate | 2% |
| Polysorbate 60 (Tween 60 from ICI) | 1% |
| Stearic acid | 1.4% |
| Triethanolamine | 0.7% |
| Carbomer | 0.4% |
| Hydrogenated polyisobutene | 12% |
| Perhydrosqualene | 12% |
| Glycerol | 5% |
| Antioxidant | 0.05% |
| Preservative | q.s. |
| Water | q.s. for 100% |

### Example 2

A depigmenting gel for the skin is prepared, which gel comprises (% by weight):

| | |
|---|---|
| Rucinol | 5% |
| Hydroxypropylcellulose | 1% |
| Antioxidant | 0.05% |
| Isopropanol | 40% |
| Preservative | q.s. |
| Water | q.s. for 100% |

1.-10. (canceled)

11. A topical composition for treating pigmentation disorders of the skin, the composition comprising, in a physiologically acceptable medium, at least 3% of rucinol or one of its salts, with respect to the total weight of the composition, wherein the rucinol or one of its salts is the sole active pharmaceutical principle in the composition.

12. The composition as defined by claim 11, wherein the rucinol or one of its salts is present in an amount ranging from 3% to 10% by weight, with respect to the total weight of the composition.

13. The composition as defined by claim 11, wherein the rucinol or one of its salts is present in an amount ranging from 3% to 5% by weight, with respect to the total weight of the composition.

14. The composition as defined by claim 11, wherein the rucinol or one of its salts is present in an amount of 5% by weight, with respect to the total weight of the composition.

15. A method of preparing a topical composition for treating pigmentation disorders of human skin, the method comprising preparing the topical composition comprising rucinol or one of its salts, as the sole active pharmaceutical principle, at a concentration of at least 3% by weight, with respect to the total weight of the composition.

16. The method as defined by claim 15, wherein the topical composition comprises rucinol or one of its salts at a concentration of 3% to 10% by weight, with respect to the total weight of the composition.

17. The method as defined by claim 15, wherein the topical composition comprises rucinol or one of its salts at a concentration of 3% to 5% by weight, with respect to the total weight of the composition.

18. The method as defined by claim 15, wherein the topical composition comprises rucinol or one of its salts at a concentration of 5% by weight, with respect to the total weight of the composition.

19. The method as defined by claim 15, wherein the pigmentation disorder is selected from the group consisting of:
melasma,
lentigines or senile lentigo,
vitiligo,
freckles,
actinic keratosis,
flat pigmented seborrhoeic warts,
postinflammatory hyperpigmentation, optionally due to infection, allergic reaction, mechanical injury, reaction to a medicament, phototoxic eruption, a scar, a trauma or inflammatory skin disease,
naevi,
genetically determined hyperpigmentation, and
hyperpigmentation of metabolic origin.

20. The composition as defined by claim 11, wherein the composition is in the form of an ointment, an aqueous solution, a lotion, a gel, a powder, an impregnated pad, a syndet, a wipe, a spray, a patch, a foam, a stick, a shampoo, a compress, a washing base, an emulsion, a cream, a salve, a suspension of microspheres or nanospheres, or a lipid or polymeric vesicle.
